(19) United States
(12) Patent Application Publication
(10) Pub. No.: US 2023/0167177 A1
(43) Pub. Date: Jun. 1, 2023

(54) NOVEL ANTI-CTLA-4 ANTIBODY POLYPEPTIDE

(71) Applicant: WUXI BIOLOGICS IRELAND LIMITED, Dublin (IE)

(72) Inventors: Yunying CHEN, Shanghai (CN); Jing LI, Shanghai (CN)

(21) Appl. No.: 16/981,806

(22) PCT Filed: Mar. 18, 2019
(86) PCT No.: PCT/CN2019/078480

§ 371 Date: Sep. 17, 2020

(30) Foreign Application Priority Data
Mar. 19, 2018 (CN) PCT/CN2018/079495

(51) Int. Cl. C07K 16/28

(52) U.S. Cl. CPC C07K 16/2818; C07K 2317/565; C07K 2317/92; C07K 2317/569; C07K 2317/24; C07K 2317/21; C07K 2317/732; C07K 2317/734; C07K 2317/33

(57) ABSTRACT

The present disclosure provides anti-CTLA-4 antibody polypeptides, polynucleotides encoding the same, pharmaceutical compositions comprising the same, and the uses thereof.

### CROSS REFERENCE TO RELATED APPLICATION

The present application is a U.S. National Stage entry of PCT Application No: PCT/CN2019/078480 filed on Mar. 18, 2019 which claims the priority to PCT Application Number PCT/CN2018/079495, filed on Mar. 19, 2018.

### FIELD OF THE INVENTION

The present disclosure generally relates to novel anti-human CTLA-4 antibody polypeptide.

### BACKGROUND

Cancer immunotherapy has become a hot research area of treating cancer. Cytotoxic T-lymphocyte-associated protein 4 (CTLA-4) is one of the validated targets of immune checkpoints. After T cell activation, CTLA-4 quickly expresses on those T cells, generally within one hour of antigen engagement with TCR. CTLA-4 can inhibit T cell signaling through competition with CD28. In addition to induced expression on activated T cells, CTLA-4 is constitutively expressed on the surface of regulatory T cells (Treg), suggesting that CTLA-4 may be required for contact-mediated suppression and associated with Treg production of immunosuppressive cytokines such as transforming growth factor beta and iterleukin-10.

CTLA-4 blockade can induce tumor regression, demonstrating in a number of preclinical and clinical studies. Two antibodies against CTLA-4 are in clinical development. Ipilimumab (MDX-010, BMS-734016), a fully human anti-CTLA-4 monoclonal antibody of IgG1-kappa isotype, is an immunomodulatory agent that has been approved as monotherapy for treatment of advanced melanoma.

A single-domain antibody (sdAb) is an antibody consisting of a single monomeric variable antibody domain. Like a whole antibody, it is able to bind selectively to a specific antigen. Single-domain antibodies are much smaller than common antibodies, which are composed of two heavy protein chains and two light chains. The first single-domain antibodies were engineered from heavy-chain antibodies found in camelids (Hamers-Casterman C, Atarhouch T, Muyldermans S, Robinson G, Hamers C, Songa E B, Bendahman N, Hamers R (1993) Naturally occurring antibodies devoid of light chains. Nature 363(6428):446-448.); these are called VHH fragments. Currently, most research into single-domain antibodies is based on heavy chain variable domains.

Single-domain antibodies have many advantages. For instance, they generally display high solubility and stability and can also be readily produced in yeast, plant, and mammalian cells (Harmsen M M, De Haard H J (2007) Properties, production, and applications of camelid single-domain antibody fragments. Appl Microbiol Biotechnol 77(1):13-22.). Further, they have good thermal stability and good tissue penetration. They are also cost efficient in production. The advantages of single-domain antibodies make them suitable for various biotechnological and therapeutic applications. For instance, they are useful in the treatment of diseases, including but not limited to cancer, infectious, inflammatory and neurodegenerative diseases.

Although antibodies against CTLA-4 are been developed, there are still spaces for improvement for antibody against CTLA-4 as a therapeutic agent. Accordingly, there is desire in the art to develop novel anti-CTLA-4 antibodies, particularly single-domain antibodies against CTLA-4.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides novel monoclonal anti-CTLA-4 antibodies, amino acid and nucleotide sequences thereof, and uses thereof.

In one aspect, the present disclosure provides an antibody polypeptide comprising a heavy chain variable domain that specifically binds to CTLA-4, wherein the heavy chain variable domain comprises:
1, 2, or 3 heavy chain complementarity determining region (CDR) sequences selected from the group consisting of: SEQ ID NO: 1, SEQ ID NO: 2, SEQ ID NO: 3 and SEQ ID NO: 10.

In certain embodiments, the heavy chain variable domain comprises a heavy chain variable region comprising SEQ ID NO: 1, SEQ ID NO: 10, and SEQ ID NO: 3. In certain embodiments, the heavy chain variable domain comprises a heavy chain variable region comprising SEQ ID NO: 1, SEQ ID NO: 2, and SEQ ID NO: 3.

In certain embodiments, the heavy chain variable domain comprises a heavy chain variable region selected from the group consisting of SEQ ID NOs: 4, 6 and 8, and a homologous sequence thereof having at least 80% sequence identity yet retaining specific binding affinity to CTLA-4.

In certain embodiments, the antibody polypeptide as provided herein further comprises one or more amino acid residue substitutions or modifications yet retaining specific binding affinity to CTLA-4.

In certain embodiments, at least one of the substitutions or modifications is in one or more of the CDR sequences, and/or in one or more of the VH sequences but not in any of the CDR sequences.

In certain embodiments, the antibody polypeptide is a single domain antibody or a heavy-chain antibody.

In certain embodiments, the heavy chain variable domain is derived from a VHH domain.

In certain embodiments, the antibody polypeptide further comprises an immunoglobulin constant region, optionally a constant region of human Ig, or optionally a constant region of human IgG.

In certain embodiments, the antibody polypeptide is isolated.

In certain embodiments, the heavy chain variable domain is of camelid origin or is humanized.

In certain embodiments, the antibody polypeptide is a nanobody.

In certain embodiments, the antibody polypeptide as provided herein is capable of specifically binding to human CTLA4 at an EC₅₀ value of no more than 0.5 nM as measured by flow cytometry.

In certain embodiments, the antibody polypeptide as provided herein is capable of blocking binding between CTLA4 and CD80 expressed on a cell surface at an IC50 value of no more than 0.15 nM, or binding between CTLA4 and CD86 expressed on a cell surface at a IC50 value of no more than 0.25 nM as measured by flow cytometry.

In certain embodiments, the antibody polypeptide as provided herein is capable of specifically binding to Cynomolgus monkey CTLA-4, and/or mouse CTLA-4.

In certain embodiments, the antibody polypeptide as provided herein, linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

In another aspect, the present disclosure provides an antibody or an antigen-binding fragment thereof, which competes for the same epitope with the antibody polypeptide of any of the preceding claims.

The present disclosure also provides a pharmaceutical composition comprising the antibody polypeptide as provided herein, the antibody or an antigen-binding fragment thereof as provided herein, and a pharmaceutically acceptable carrier.

The present disclosure also provides a polynucleotide encoding the antibody polypeptide as provided herein. In certain embodiments, the polynucleotide is isolated.

In certain embodiments, the polynucleotide as provided herein comprises a nucleotide sequence selecting from a group consisting of SEQ ID NO: 5, SEQ ID NO: 7 and SEQ ID NO: 9, and/or a homologous sequence thereof having at least 80% (e.g. at least 85%, 88%, 90%, 92%, 93%, 94%, 95%, 96%, 97%, 98%, or 99%) sequence identity, and/or a variant thereof having only degenerate substitutions.

The present disclosure also provides a vector comprising the polynucleotide as provided herein.

The present disclosure also provides a host cell comprising the vector as provided herein.

The present disclosure also provides a method of expressing the antibody polypeptide as provided herein, comprising culturing the host cell as provided herein under the condition at which the vector as provided herein is expressed.

The present disclosure also provides a method of treating a disease or condition in a subject that would benefit from modulation of CTLA-4 activity, comprising administering to the subject a therapeutically effective amount of the antibody polypeptide of any as provided herein or the pharmaceutical composition as provided herein.

In certain embodiments, the disease or condition is a CTLA-4 related disease or condition.

In certain embodiments, the disease or condition is cancer, autoimmune disease, inflammatory disease, infectious disease, graft versus host disease (GVHD), or transplant rejection.

In certain embodiments, the cancer is lymphoma, bladder cancer, bone cancer, brain and central nervous system cancer, breast cancer, uterine or endometrial cancer, rectal cancer, esophageal cancer, head and neck cancer, anal cancer, gastrointestinal cancer, intra-epithelial neoplasm, kidney or renal cancer, leukemia, liver cancer, lung cancer, melanoma, myeloma, pancreatic cancer, prostate cancer, sarcoma, skin cancer, squamous cell cancer, stomach cancer, testicular cancer, vulval cancer, cancer of the endocrine system, cancer of the parathyroid gland, cancer of the adrenal gland, penile carcinoma, solid tumors of childhood, tumor angiogenesis, spinal axis tumor, pituitary adenoma, or epidermoid cancer.

In certain embodiments, the disease or condition is an environmentally induced cancer induced by asbestos or hematologic malignancies, wherein said cancer is selected from multiple myeloma, B-cell lymphoma, Hodgkin lymphoma, primary mediastinal B-cell lymphoma, non-Hodgkin's lymphoma, acute myeloid lymphoma, chronic myelogenous leukemia, chronic lymphoid leukemia (CLL), follicular lymphoma, diffuse large B-cell lymphoma (DLBCL), Burkitt's lymphoma, immunoblastic large cell lymphoma, precursor B-lymphoblastic lymphoma, mantle cell lymphoma, acute lymphoblastic leukemia (ALL), mycosis fungoides, anaplastic large cell lymphoma, T-cell lymphoma, and precursor T-lymphoblastic lymphoma, and any combinations of said cancers.

In certain embodiments, the subject is human.

In certain embodiments, the administration is via oral, nasal, intravenous, subcutaneous, sublingual, or intramuscular administration.

In another aspect, the disclosure also provides a method of modulating CTLA-4 activity in a CTLA-4-expressing cell, comprising exposing the CTLA-4-expressing cell to the antibody polypeptide as provided herein.

The present disclosure also provides a method of detecting presence or amount of CTLA-4 in a sample, comprising contacting the sample with the antibody polypeptide as provided herein, and determining the presence or the amount of CTLA-4 in the sample.

The present disclosure also provides herein a method of diagnosing a CTLA-4 related disease or condition in a subject, comprising: a) contacting a sample obtained from the subject with the antibody polypeptide of any as provided herein; b) determining presence or amount of CTLA-4 in the sample; and c) correlating the presence or the amount of CTLA-4 to existence or status of the CTLA-4 related disease or condition in the subject.

The present disclosure also provides use of the antibody polypeptide as provided herein in the manufacture of a medicament for treating a CTLA-4 related disease or condition in a subject.

The present disclosure also provides use of the antibody polypeptide as provided herein in the manufacture of a diagnostic reagent for diagnosing a CTLA-4 related disease or condition.

The present disclosure also provides a kit comprising the antibody polypeptide as provided herein useful in detecting CTLA-4.

### BRIEF DESCRIPTION OF FIGURES

FIG. 1A shows that W3166-z13 and W3166-z17 bind to cell surface human CTLA4 as measured by FACS assay.

FIG. 1B shows that W3166-z13 and W3166-z17 bind to human CTLA4 as measured by ELISA assay.

FIG. 2A shows that W3166-z13 and W3166-z17 bind to cell surface cynomolgus CTLA4 as measured by FACS assay.

FIG. 2B shows that W3166-z13 and W3166-z17 bind cynomolgus CTLA4 as measured by ELISA assay.

FIG. 3A shows that W3166-z13 and W3166-z17 block CD80 binding to human CTLA4 as measured by ELISA assay.

FIG. 3B shows that W3166-z13 and W3166-z17 block CD86 binding to human CTLA4 as measured by ELISA assay.

FIG. 4A shows that W3166-z13 and W3166-z17 block CD80 binding to cell surface human CTLA4 more effectively than W316-BMK1 as measured by FACS assay.

FIG. 4B shows that W3166-z13 and W3166-z17 block CD86 binding to cell surface human CTLA4 more effectively than W316-BMK1 as measured by FACS assay.

FIG. 5A shows that W3166-z13 and W3166-z17 enhance IFN-γ production more potently than W316-BMK1 in human allogeneic MLR assay.

FIG. 5B shows that W3166-z13 and W3166-z17 enhance IL-2 production in human allogeneic MLR in a dose-dependent manner. The potency is comparable to W316-BMK1.

FIG. 6 shows that W3166-z13 and W3166-z17 share similar epitope bin as W316-BMK1 as measured by ELISA epitope binning test.

FIG. 7 shows that W3166-z13 and W3166-z17 induce ADCC effect on human CTLA4 transfected cells.

FIG. 8 shows that W3166-z13 and W3166-z17 do not induce CDC effect on human CTLA4 transfected cells.

FIG. 9 shows W3166-z13 and W3166-z17 are stable in human serum stability test, as measured by FACS assay.

FIG. 10 shows W3166-z13 and W3166-z17 specifically bind to human CTLA-4 and do not cross-react with hICOS, BTLA, hCD28 and hPD1, as measured by ELISA assay

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, or monovalent antibody that binds to a specific antigen. The term “antibody” as used herein intends to encompass broadly to both conventional four-chain antibodies and also less-conventional antibodies that do not have four chains (such as antibodies naturally devoid of light chains).

A conventional intact antibody is a heterotetramer comprising two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The conventional antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of Immunological Interest, 5^{th} Ed. Public Health Service, National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

Unlike conventional antibodies which are heterotetramers, there are homodimeric immunoglobulins and are naturally devoid of light chains. Such antibodies are found in, for example, camelids (camel, dromedary, llama, alpaca, etc.), and are also called heavy-chain antibodies with a molecular weight of about 80 kD (Hamers-Casterman C. et al., 1993, Nature, 363:446-448).

The term “antibody polypeptide” as used herein refers to an antigen-binding protein or polypeptide comprising an antibody fragment (such as a CDR, and/or a variable region sequence). An antibody polypeptide can comprise or can be, for example, a heavy-chain antibody (a VHH antibody), a variable domain of a heavy-chain antibody, a VHH domain, or a single domain antibody containing a single variable domain. The antibody polypeptide may further comprise additional domains such as a constant region, an Fc domain, and/or a second variable domain specifically binding to a different antigen or different epitope.

“Heavy-chain antibody” and “VHH antibody” are used interchangeably herein, and refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Although devoid of light chains, heavy chain antibodies have an authentic antigen-binding repertoire (Hamers-Casterman C. et al., 1993, Nature, 363:446-448; Nguyen V K. et al., 2002, Immunogenetics, 54(1):39-47; Nguyen V K. et al., 2003, Immunology, 109(1):93-101).

“VHH domain” as used herein refers to the heavy chain variable domain derived from a heavy-chain antibody. VHH domain represents the smallest known antigen-binding unit generated by adaptive immune responses (Koch-Nolte F. et al., 2007, FASEB J., 21(13):3490-8. Epub 2007 June 15).

A “single domain antibody” refers to an antibody fragment containing only a single variable region of a heavy chain or a single variable region of a light chain. In certain embodiments, the single domain antibody has or consists of only a single heavy-chain variable domain of a heavy-chain antibody.

A “nanobody” refers to an antibody fragment that consists of a VHH domain from a heavy chain antibody and two constant domains, CH2 and CH3.

In certain instances, two or more VHH domains can be covalently joined with a peptide linker to create a bivalent or multivalent domain antibody. The two VHH domains of a bivalent domain antibody may target the same or different antigens.

The term “bivalent” as used herein refers to an antibody or antibody polypeptide having two antigen-binding sites; the term “monovalent” refers to an antibody or antibody polypeptide having only one single antigen-binding site; and the term “multivalent” refers to an antibody or antibody polypeptide having multiple antigen-binding sites. In some embodiments, the antibody or antibody polypeptide is bivalent.

The term “chimeric” as used herein, means an antibody or antibody polypeptide having a portion of heavy chain derived from one species, and the rest of the heavy chain derived from a different species. In an illustrative example, a chimeric antibody may comprise a constant region derived from human and a variable region from a non-human animal, such as from camelidae. In some embodiments, the non-human animal is a mammal, for example, a camelidae, a mouse, a rat, a rabbit, a goat, a sheep, a guinea pig, or a hamster.

The term “humanized” as used herein means that the antibody or antibody polypeptide comprises CDRs derived from non-human animals, FR regions derived from human, and when applicable, the constant regions derived from human.

“CTLA-4” as used herein, refers to the Cytotoxic T-lymphocyte-associated protein 4 derived from any vertebrate source, including mammals such as primates (e.g. humans, monkeys) and rodents (e.g., mice and rats). Exemplary sequence of human CTLA-4 includes human CTLA-4 protein (NCBI Ref Seq No. AAL07473.1). Exemplary sequence of CTLA-4 includes Macaca fascicularis (monkey) CTLA-4 protein (NCBI Ref Seq No. XP_005574071.1).

The term “CTLA-4” as used herein is intended to encompass any form of CTLA-4, for example, 1) native unprocessed CTLA-4 molecule, “full-length” CTLA-4 chain or naturally occurring variants of CTLA-4, including, for example, splice variants or allelic variants; 2) any form of CTLA-4 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of CTLA-4 subunit generated through recombinant method.

The term “anti-CTLA-4” antibody polypeptide refers to an antibody polypeptide that is capable of specific binding CTLA-4 (e.g. human or monkey CTLA-4).

The term “specific binding” or “specifically binds” as used herein refers to a non-random binding reaction between two molecules, such as for example between an antibody and an antigen. In certain embodiments, the antibody polypeptides provided herein specifically bind to human CTLA-4 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, 2×10⁻⁸ M, ≤10⁻⁸ M, ≤5×10⁻⁹ M, ≤4×10⁻⁹M, ≤3×10⁻⁹M, ≤2×10⁻⁹ M, or 10⁻⁹ M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The ability to “block binding” or “compete for the same epitope” as used herein refers to the ability of an antibody polypeptide to inhibit the binding interaction between two molecules (e.g. human CTLA-4 and an anti-CTLA-4 antibody) to any detectable degree. In certain embodiments, an antibody polypeptide that blocks binding between two molecules inhibits the binding interaction between the two molecules by at least 85%, or at least 90%. In certain embodiments, this inhibition may be greater than 85%, or greater than 90%.

The term “epitope” as used herein refers to the specific group of atoms or amino acids on an antigen to which an antibody binds. Two antibodies may bind the same or a closely related epitope within an antigen if they exhibit competitive binding for the antigen. For example, if an antibody polypeptide blocks binding of a reference antibody to the antigen by at least 85%, or at least 90%, or at least 95%, then the antibody polypeptide may be considered to bind the same/closely related epitope as the reference antibody.

Those skilled in the art will recognize that it is possible to determine, without undue experimentation, if a given antibody binds to the same epitope as the antibody of present disclosure (e.g., camelid VHH antibody W3166, and humanized antibody W3166-z13 and W3166-z17) by ascertaining whether the former prevents the latter from binding to a CTLA-4 antigen polypeptide. If the given antibody competes with the antibody of present disclosure, as shown by a decrease in binding by the antibody of present disclosure to the CTLA-4 antigen polypeptide, then the two antibodies bind to the same, or a closely related, epitope. Or if the binding of a given antibody to the CTLA-4 antigen polypeptide was inhibited by the antibody of present disclosure, then the two antibodies bind to the same, or a closely related, epitope.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

The term “homolog” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody polypeptide” refers to the antibody polypeptide having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody polypeptide, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

The phrase “host cell” as used herein refers to a cell into which an exogenous polynucleotide and/or a vector has been introduced.

A “CTLA-4-related” disease or condition as used herein refers to any disease or condition caused by, exacerbated by, or otherwise linked to increased or decreased expression or activities of CTLA-4. In some embodiments, the CTLA-4 related condition is immune-related disorder, such as, for example, cancer, autoimmune disease, inflammatory disease or infectious disease, graft versus host disease (GVHD), or transplant rejection.

“Cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Anti-CTLA-4 Antibody Polypeptide

The present disclosure provides anti-CTLA-4 antibody polypeptides comprising one or more (e.g. 1, 2, or 3) CDR sequences of an anti-CTLA-4 single domain antibody W3166.

“W3166” as used herein refers to a VHH antibody having a heavy chain variable region comprising the sequence of SEQ ID NO: 4.

“W3166-z13” as used herein refers to a humanized VHH antibody based on W3166 that comprises a heavy chain variable region comprising the sequence of SEQ ID NO: 6. W3166-z13 has comparable affinity to the antigen as compared with its parent antibody W3166.

“W3166-z17” as used herein refers to a humanized VHH antibody based on W3166 that comprises a heavy chain variable region comprising the sequence of SEQ ID NO: 8. W3166-z17 has comparable affinity to the antigen as compared with its parent antibody W3166.

Table 1 shows the CDR sequences of the anti-CTLA-4 single domain antibody. The heavy chain variable region sequences are also provided below in Table 2 and Table 3.

TABLE 1
CDR amino acid sequences

| | | CDR1 | CDR2 | CDR3 |
|---|---|---|---|---|
| W3166 | HCDR | SEQ ID | SEQ ID | SEQ ID |
| | | NO: 1 | NO: 2 | NO: 3 |
| | | GRTFSS | SIRWSDNTT | GPTRLSFYS |
| | | YAMG | YVPNSVKG | GNYRTYDS |
| W3166-z13, | HCDR | SEQ ID | SEQ ID | SEQ ID |
| W3166-Z17 | | NO: 1 | NO: 10 | NO: 3 |
| | | GRTFSS | SIRWSDQTT | GPTRLSFYS |
| | | YAMG | YVPNSVKG | GNYRTYDS |

TABLE 2
Variable region amino acid sequences

| | VH |
|---|---|
| W3166 | SEQ ID NO: 4 |
| | QVQLVESGGGLVQAGGSLRLSCAAS [symbol] W |
| | FRQAPGMEREFVA [symbol] RFTISRD |
| | NAKNTVYLQMNTLKPEDTAVYYCAT [symbol] |
| | [symbol] WGQGTLVTVSS |
| W3166- | SEQ ID NO: 6 |
| z13 | QVQLVESGGGLVQPGGSLRLSCAAS [symbol] W |
| | FRQAPGMEREFVA [symbol] RFTISRD |
| | NSKNTLYLOMNSLRPEDTAVYYCAT [symbol] |
| | [symbol] WGQGTLVTVSS |
| W3166- | SEQ ID NO: 8 |
| z17 | QVQLVESGGGVVQPGGSLRLSCAAS [symbol] W |
| | FRQAPGKEREFVA [symbol] RFTISRDN |
| | SKNTLYLOMNSLRPEDTAVYYCAT [symbol] |
| | [symbol] WGQGTLVTVSS |

TABLE 3
Variable region nucleotide sequences

| | VHnu |
|---|---|
| W3166 | SEQ ID NO: 5 |
| | caggtgcagctcgtggagtctgggggaggattggtg |
| | caggctgggggctctctgagactctcctgtgcggcc |
| | tctggacgcaccttcagtagctatgccatgggctgg |
| | ttccgccaggctccagggatggagcgtgagtttgta |
| | gcatctattaggtggagtgataatacgacatacgtc |
| | cctaactccgtgaagggccgattcaccatctccaga |
| | gacaacgccaagaacacggtgtatctgcaaatgaac |
| | accctgaaacctgaggacacggccgtttattactgt |
| | gcaacagggcccacgagactatcattttatagtggt |
| | aattatagaacttatgactcctggggccaggggacc |
| | ctggtcaccgtctcctca |
| W3166- | SEQ ID NO: 7 |
| z13 | caggtgcagctggtggagagcggaggcggactggtg |
| | cagcctggaggaagcctgagactgagctgcgccgcc |
| | agcggcagaaccttcagcagctacgccatgggctgg |
| | ttcagacaggcccctggcatggagagagagttcgtg |
| | gccagcatcaggtggtccgaccagaccacctacgtg |
| | cccaacagcgtgaagggcaggttcaccatcagcagg |
| | gacaacagcaagaacaccctgtacctccagatgaac |
| | agcctgagacccgaggataccgccgtgtactattgc |
| | gccaccggccccaccagactgagcttctacagcggc |
| | aactacaggacctacgacagctggggccagggaacc |
| | ctggtgaccgtgagcagc |
| W3166- | SEQ ID NO: 9 |
| z17 | caggtgcagctggtggagagcggaggcggagtggtg |
| | cagcctggaggaagcctgagactgagctgcgccgcc |
| | agcggcagaaccttcagcagctacgccatgggctgg |
| | ttcagacaggcccctggcaaggagagagagttcgtg |
| | gccagcatcaggtggtccgaccagaccacctacgtg |
| | cccaacagcgtgaagggcaggttcaccatcagcagg |
| | gacaacagcaagaacaccctgtacctccagatgaac |
| | agcctgagacccgaggataccgccgtgtactattgc |
| | gccaccggccccaccagactgagcttctacagcggc |
| | aactacaggacctacgacagctggggccagggaacc |
| | ctggtgaccgtgagcagc |

In certain embodiments, the antibody polypeptides provided herein are single domain antibodies.

In certain embodiments, the heavy chain variable domain of the antibody polypeptides provided herein is derived from a VHH domain. VHH domains are heavy chain variable domains derived from antibodies naturally devoid of light chains, for example, antibodies derived from Camelidae species (see, e.g. WO9404678), for example in camel, llama, dromedary, alpaca and guanaco. VHH domains are single polypeptides, and are stable.

In certain embodiments, the heavy chain variable domain of the antibody polypeptides provided herein is of camelid origin.

CDRs are known to be responsible for antigen binding, however, it has been found that not all of the 6 CDRs are indispensable or unchangeable. In other words, it is possible to replace or change or modify one or more CDRs in anti-CTLA-4 single domain antibody W3166, yet substantially retain the specific binding affinity to CTLA-4.

In certain embodiments, the anti-CTLA-4 antibody polypeptides provided herein comprise a heavy chain CDR3 sequence of W3166. In certain embodiments, the anti-CTLA-4 antibody polypeptides provided herein comprise a heavy chain CDR3 sequence of SEQ ID NO: 3. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In certain embodiments, the antibody polypeptides provided herein comprise suitable framework region (FR) sequences, as long as the antibody polypeptides can specifically bind to CTLA-4. The CDR sequences provided in Table 1 are obtained from camelid antibodies, but they can be grafted to any suitable FR sequences of any suitable species such as mouse, human, rat, rabbit, among others, using suitable methods known in the art such as recombinant techniques.

In certain embodiments, the antibody polypeptides provided herein are humanized. A humanized antibody polypeptide is desirable in its reduced immunogenicity in human. A humanized antibody polypeptide is chimeric in its variable regions, as non-human CDR sequences are grafted to human or substantially human FR sequences. Humanization of an antibody polypeptide can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain variable domains can be selected to achieve this purpose using methods known in the art. In an illustrative example, “best-fit” approach can be used, where anon-human (e.g. camelid) antibody variable domain sequence is screened or BLASTed against a database of known human variable domain sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et at. (1992) Proc. Natl. Acad. Sci. USA, 89:4285; Presta et al. (1993) J. Immunol., 151:2623).

In certain embodiments, the humanized antibody polypeptides provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody. In some embodiments, the humanized antibody polypeptide comprise human FR1-4.

In certain embodiments, the humanized antibody polypeptides provided herein comprise one or more FR sequences of W3166-z13, or W3166-z17.

The two exemplary humanized anti-CTLA-4 single domain antibodies W3166-z13 and W3166-z17 both retained the specific binding affinity to CTLA-4, and are at least comparable to, or even better than, the parent camelid antibodies in that aspect.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody polypeptides closely approximate the non-human parent antibody structure. In certain embodiments, the humanized antibody polypeptides provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains.

In certain embodiments, the antibody polypeptide provided herein comprises a heavy chain variable domain sequence selected from the group consisting of SEQ ID NO: 4, SEQ ID NO: 6, and SEQ ID NO: 8.

In some embodiments, the anti-CTLA-4 antibody polypeptide provided herein comprises all or a portion of the heavy chain variable domain. In one embodiment, the anti-CTLA-4 antibody polypeptides provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-CTLA-4 antibody polypeptide provided herein further comprises an immunoglobulin constant region. In some embodiments, an immunoglobulin constant region comprises a heavy chain. The heavy chain constant region comprises CHL hinge, and/or CH2-CH3 regions. In certain embodiments, the heavy chain constant region comprises an Fc region. In certain embodiments, the heavy chain constant region comprises or is a CH2-CH3 region.

In some embodiments, the anti-CTLA-4 antibody polypeptide provided herein has a constant region of an immunoglobulin (Ig), optionally a human Ig, optionally a human IgG. The constant region can be in any suitable isotype. In certain embodiments, the anti-CTLA-4 antibody polypeptide provided herein comprises a constant region of IgG1 isotype, which could induce ADCC or CDC, or a constant region of IgG4 or IgG2 isotype, which has reduced or depleted effector function. Effector functions such as ADCC and CDC can lead to cytotoxicity to cells expressing CTLA-4. Effector functions can be evaluated using various assays such as Fc receptor binding assay, C1q binding assay, and cell lysis assay.

Binding affinity of the antibody polypeptide provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g. K_{D}) can be appropriately determined using suitable methods known in the art, including, for example, flow cytometry assay. In some embodiments, binding of the antibody polypeptide to the antigen at different concentrations can be determined by flow cytometry, the determined mean fluorescence intensity (MFI) can be firstly plotted against antibody concentration, K_{D} value can then be calculated by fitting the dependence of specific binding fluorescence intensity (Y) and the concentration of antibodies (X) into the one site saturation equation: Y=Bₘₐₓ*X/(K_{D}+X) using Prism version 5 (GraphPad Software, San Diego, Calif.), wherein Bₘₐₓ refers to the maximum specific binding of the tested antibody polypeptide to the antigen.

In some embodiments, the anti-CTLA-4 antibody polypeptides provided herein are capable of specifically binding to human CTLA-4 with a binding affinity (K_{D}) of no more than 5×10⁻¹¹M, no more than 1×10⁻¹⁰M, no more than 5×10⁻¹⁰M, no more than 1×10⁻⁹M, no more than 5×10⁻⁹M as measured by flow cytometry assay.

Binding of the antibody polypeptides to human CTLA-4 can also be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assay. In certain embodiments, the antibody polypeptides provided herein specifically bind to human CTLA-4 at an EC₅₀ (i.e. 50% binding concentration) of no more than 0.5 nM, no more than 1 nM, no more than 2 nM by flow cytometry assay.

In certain embodiments, the anti-CTLA-4 antibody polypeptides provided herein cross-react with Cynomolgus monkey CTLA-4.

In certain embodiments, the antibody polypeptides bind to Cynomolgus monkey CTLA-4 with a binding affinity similar to that of human CTLA-4. For example, binding of the exemplary single domain antibodies W3166, W3166-z13, or W3166-z17 to Cynomolgus monkey CTLA-4 is at a similar K_{D} or EC₅₀ value to that of human CTLA-4.

In certain embodiments, the antibody polypeptides provided herein specifically bind to Cynomolgus monkey CTLA-4 with a K_{D} of no more than 0.1 nM, no more than 0.5 nM, no more than 1 nM by flow cytometry assay, or with an EC₅₀ of no more than 10 nM, no more than 5 nM, no more than 2 nM or no more than 1.2 nM by flow cytometry assay.

In certain embodiments, the antibody polypeptides provided herein have a specific binding affinity to human CTLA-4 which is sufficient to provide for diagnostic and/or therapeutic use.

In certain embodiments, the antibody polypeptides provided herein block binding of human CTLA-4 to its ligand CD80 and CD86, thereby providing biological activity including, for example, inducing cytokine production from the activated T cells (such as CD4+ T cells and CD8+ T cells), inducing proliferation of activated T cells (such as CD4+ T cells and CD8⁺ T cells), and reversing T reg's suppressive function. Exemplary cytokines include IL-2 and IFNγ. The cytokine production can be determined using methods known in the art, for example, by ELISA. Methods can also be used to detect proliferation of T cells, including [³H] thymidine incorporation assay.

The antibody polypeptides provided herein can be monoclonal, humanized, chimeric, recombinant, labeled, bivalent, or anti-idiotypic. A recombinant antibody polypeptide is an antibody polypeptide prepared in vitro using recombinant methods rather than in animals.

Variants

The antibody polypeptides provided herein also encompass various variants thereof. In certain embodiments, the antibody polypeptides encompasses various types of variants of an exemplary antibody provided herein, i.e., W3166, W3166-z13, and W3166-z17.

In certain embodiments, the antibody polypeptide variants comprise one or more modifications or substitutions in one or more CDR sequences as provided in Table 1, one or more variable region sequences (but not in any of the CDR sequences) provided in Table 2, and/or the constant region (e.g. Fc region). Such variants retain specific binding affinity to CTLA-4 of their parent antibodies, but have one or more desirable properties conferred by the modification(s) or substitution(s). For example, the antibody polypeptide variants may have improved antigen-binding affinity, improved productivity, improved stability, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibody polypeptides are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

Affinity Variant

Affinity variant may contain modifications or substitutions in one or more CDR sequences as provided in Table 1, one or more FR sequences, or the heavy chain variable region sequences provided in Table 2. FR sequences can be readily identified by a skilled person in the art based on the CDR sequences in Table 1 and variable region sequences in Table 2, as it is well-known in the art that a CDR region is flanked by two FR regions in the variable region. The affinity variants retain specific binding affinity to CTLA-4 of the parent antibody, or even have improved CTLA-4 specific binding affinity over the parent antibody. In certain embodiments, at least one (or all) of the substitution(s) in the CDR sequences, FR sequences, or variable region sequences comprises a conservative substitution.

A skilled artisan will understand that in the CDR sequences and variable region sequences provided in Table 1 and Table 2, one or more amino acid residues may be substituted yet the resulting antibody polypeptide still retain the binding affinity to CTLA-4, or even have an improved binding affinity. Various methods known in the art can be used to achieve this purpose. For example, a library of antibody variants (such as Fab or scFv variants) can be generated and expressed with phage display technology, and then screened for the binding affinity to human CTLA-4. For another example, computer software can be used to virtually simulate the binding of the antibodies to human CTLA-4, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody polypeptides provided herein comprise one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in the CDR sequences and/or FR sequences in total.

In certain embodiments, the anti-CTLA-4 antibody polypeptides comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to CTLA-4 at a level similar to or even higher than its parent antibody.

In certain embodiments, the anti-CTLA-4 antibody polypeptides comprise one or more variable region sequences having at least 80% (e.g. at least 85%, 88%, 90%, 91%, 92%, 93%, 94%, 95%, 96%, 97%, 98%, 99%) sequence identity to that (or those) listed in Table 2, and in the meantime retain the binding affinity to CTLA-4 at a level similar to or even higher than its parent antibody. In some embodiments, a total of 1 to 10 amino acids have been substituted, inserted, or deleted in a variable region sequence listed in Table 2. In some embodiments, the substitutions, insertions, or deletions occur in regions outside the CDRs (e.g., in the FRs).

Glycosylation Variant

The anti-CTLA-4 antibody polypeptides provided herein also encompass a glycosylation variant, which can be obtained to either increase or decrease the extent of glycosylation of the antibody polypeptide.

The antibody polypeptide may comprise one or more amino acid residues with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue. In certain embodiments, the heavy chain CDR2 of the antibody provided herein comprise an N55Q substitution (kabat numbering), such that the potential glycosylation site is removed.

Cysteine-Engineered Variant

The anti-CTLA-4 antibody polypeptides provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibody polypeptides to introduce free cysteine residues are known in the art, see, for example, WO2006/034488.

Fc Variant

The anti-CTLA-4 antibody polypeptides provided herein also encompass an Fc variant, which comprises one or more amino acid residue modifications or substitutions at its Fc region and/or hinge region.

In certain embodiments, the anti-CTLA-4 antibody polypeptides comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody polypeptide to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

In certain embodiments, the anti-CTLA-4 antibody polypeptides comprise one or more amino acid substitution(s) that alters the antibody-dependent cellular cytotoxicity (ADCC). Certain amino acid residues at the Fc region (e.g. at the CH2 domain) can be substituted to provide for altered (e.g. enhanced, decreased, or depleted) ADCC activity. Alternatively or additionally, carbohydrate structures on the antibody can be changed to alter (e.g. enhance, decrease or deplete) ADCC activity. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O., et al., Mol Cancer Ther. 2008, 7(8): 2517-27; Shields R. L. et al, J. Biol. Chem, 2002, 277: 26733-26740; Shinkawa T. et al, J. Biol. Chem, 2003, 278: 3466-3473.

In certain embodiments, the anti-CTLA-4 antibody polypeptides comprise one or more amino acid substitution(s) that alters Complement Dependent Cytotoxicity (CDC), for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants.

In certain embodiments, the anti-CTLA-4 antibody polypeptides comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

Various techniques can be used for the production of VHH or single domain antibodies. For example, VHHs may be obtained using methods known in the art such as by immunizing a camel and obtaining hybridomas therefrom, or by cloning a library of single domain antibodies using molecular biology techniques known in the art and subsequent selection by using phage display.

In another aspect of the present disclosure, an antibody polypeptide provided herein may comprise two or more single domain antibodies which have been joined. The single domain antibodies may be identical in sequence and directed against the same target or antigen. Depending on the number of VHHs linked, the antibody polypeptide may be bivalent (2 VHHs), trivalent (3 VHHs), tetravalent (4 VHHs) or have a higher valency molecules.

Conjugates

In some embodiments, the anti-CTLA-4 antibody polypeptides further comprise a conjugate moiety. The conjugate moiety can be linked to the antibody polypeptides. A conjugate moiety is a non-proteinaceous moiety that can be attached to the antibody polypeptide. It is contemplated that a variety of conjugate moieties may be linked to the antibody polypeptides provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibody polypeptides by covalent binding, affinity binding, intercalation, coordinate binding, complexation, association, blending, or addition, among other methods.

In certain embodiments, the antibody polypeptides disclosed herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies may be linked to a conjugate moiety indirectly, or through another conjugate moiety. For example, the antibody polypeptides may be conjugated to biotin, then indirectly conjugated to a second conjugate that is conjugated to avidin. The conjugate can be a clearance-modifying agent, a toxin (e.g., a chemotherapeutic agent), a detectable label (e.g., a radioactive isotope, a lanthanide, a luminescent label, a fluorescent label, or an enzyme-substrate label), or purification moiety.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection.

In certain embodiments, the conjugate moiety can be a clearance-modifying agent which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibody polypeptides provided herein is used for a base for a conjugate.

Polynucleotides and Recombinant Methods

The present disclosure provides polynucleotides that encode the anti-CTLA-4 antibody polypeptides.

The term “nucleic acid” or “polynucleotide” as used herein refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In certain embodiments, the polynucleotides comprise one or more nucleotide sequences as shown in SEQ ID NOs: 5, 7, 9 (e.g. at least 85%, 88%, 90%, 92%, 93%, 94%, 95%, 96%, 97%, 98%, or 99%), and/or a homologous sequence thereof having at least 80% sequence identity, and/or a variant thereof having only degenerate substitutions, and encodes the variable region of the exemplary antibodies provided herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-CTLA-4 antibody polypeptides (e.g. including the sequences as shown in Table 3) can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors (e.g., expression vectors) containing the nucleic acid sequence provided herein encoding the antibody polypeptides, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody polypeptide can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for expressing anti-CTLA-4 antibody polypeptides. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L−1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is 293F cell.

Host cells are transformed with the above-described expression or cloning vectors for anti-CTLA-4 antibody polypeptide production and cultured in conventional nutrient media modified as appropriate for inducing promoters, selecting transformants, or amplifying the genes encoding the desired sequences. In another embodiment, the antibody polypeptides may be produced by homologous recombination known in the art.

The host cells used to produce the antibody polypeptides provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody polypeptides can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CTLA-4 antibody polypeptides prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody polypeptide. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Pharmaceutical Composition

The present disclosure further provides pharmaceutical compositions comprising an anti-CTLA-4 antibody polypeptide provided herein and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody polypeptide and conjugates as provided herein decreases oxidation of the antibody polypeptide. This reduction in oxidation prevents or reduces loss of binding affinity, thereby improving antibody stability and maximizing shelf-life. Therefore, in certain embodiments compositions are provided that comprise one or more antibody polypeptides as disclosed herein and one or more antioxidants such as methionine. Further provided are methods for preventing oxidation of, extending the shelf-life of, and/or improving the efficacy of an antibody polypeptide as provided herein by mixing the antibody polypeptide with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody polypeptide as disclosed herein in a suitable solvent. The solvent may contain an excipient which improves the stability or other pharmacological components of the powder or reconstituted solution, prepared from the powder. Excipients that may be used include, but are not limited to, water, dextrose, sorbitol, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CTLA-4 antibody polypeptides or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

Methods of Use

The present disclosure also provides therapeutic methods comprising: administering a therapeutically effective amount of the antibody polypeptides as provided herein to a subject in need thereof, thereby treating or preventing a CTLA-4-related condition or a disorder. In some embodiment, the CTLA-4-related condition or a disorder is cancer, autoimmune disease, inflammatory disease, infectious disease, graft versus host disease (GVHD), or transplant rejection.

Examples of cancer include but are not limited to, lymphoma, bladder cancer, bone cancer, brain and central nervous system cancer, breast cancer, uterine or endometrial cancer, rectal cancer, esophageal cancer, head and neck cancer, anal cancer, gastrointestinal cancer, intra-epithelial neoplasm, kidney or renal cancer, leukemia, liver cancer, lung cancer (e.g. non-small cell lung cancer and small cell lung cancer), melanoma, myeloma, pancreatic cancer, prostate cancer, sarcoma, skin cancer, squamous cell cancer, stomach cancer, testicular cancer, vulval cancer, cancer of the endocrine system, cancer of the parathyroid gland, cancer of the adrenal gland, penile carcinoma, solid tumors of childhood, tumor angiogenesis, spinal axis tumor, pituitary adenoma, or epidermoid cancer.

Examples of autoimmune diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

Examples of infectious disease include, but are not limited to, fungus infection, parasite/protozoan infection or chronic viral infection, for example, malaria, coccidioiodmycosis immitis, histoplasmosis, onychomycosis, aspergilosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV, cytomegalovirus, herpes simplex virus type I, herpes simplex virus type II, human papilloma virus, adenovirus, human immunodeficiency virus I, human immunodeficiency virus II, Kaposi West sarcoma associated herpes virus epidemics, thin ring virus (Torquetenovirus), human T lymphotrophic viruse I, human T lymphotrophic viruse II, varicella zoster, JC virus or BK virus.

The therapeutically effective amount of an antibody polypeptide as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the antibody polypeptides as provided herein may be administered at a therapeutically effective dosage of about 0.01 mg/kg to about 100 mg/kg. In certain of these embodiments, the antibody polypeptide is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibody polypeptides disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibody polypeptides disclosed herein may be administered alone or in combination with one or more additional therapeutic means or agents. For example, the antibody polypeptides disclosed herein may be administered in combination with another therapeutic agent, for example, a chemotherapeutic agent or an anti-cancer drug.

In certain of these embodiments, an antibody polypeptide as disclosed herein that is administered in combination with one or more additional therapeutic agents may be administered simultaneously with the one or more additional therapeutic agents, and in certain of these embodiments the antibody polypeptide and the additional therapeutic agent(s) may be administered as part of the same pharmaceutical composition. However, an antibody polypeptide administered “in combination” with another therapeutic agent does not have to be administered simultaneously with or in the same composition as the agent. An antibody polypeptide administered prior to or after another agent is considered to be administered “in combination” with that agent as the phrase is used herein, even if the antibody polypeptide and second agent are administered via different routes. Where possible, additional therapeutic agents administered in combination with the antibody polypeptides disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of using the anti-CTLA-4 antibody polypeptides.

In some embodiments, the present disclosure provides methods of detecting presence or amount of CTLA-4 in a sample, comprising contacting the sample with the antibody polypeptide, and determining the presence or the amount of CTLA-4 in the sample.

In some embodiments, the present disclosure provides methods of diagnosing a CTLA-4 related disease or condition in a subject, comprising: a) contacting a sample obtained from the subject with the antibody polypeptide provided herein; b) determining presence or amount of CTLA-4 in the sample; and c) correlating the existence of the CTLA-4 to the CTLA-4 related disease or condition in the subject.

In some embodiments, the present disclosure provides kits comprising the antibody polypeptide provided herein, optionally conjugated with a detectable moiety. The kits may be useful in detection of CTLA-4 or diagnosis of CTLA-4 related disease.

In some embodiments, the present disclosure also provides use of the antibody polypeptide provided herein in the manufacture of a medicament for treating a CTLA-4 related disease or condition in a subject, in the manufacture of a diagnostic reagent for diagnosing a CTLA-4 related disease or condition.

### ADVANTAGES

The antibody polypeptides provided herein are advantageous over existing therapies in many aspects. For example, the antibody polypeptides provided herein have better affinity to cell surface human CTLA4, and is more effective at blocking CTLA4 binding to cell surface CD80 and CD86, as compared with ipilumumab, and is more effective at inducing ADCC effect on hCTLA4 transfected cells.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1: Materials and Methods

1.1 Protein Preparation

1.1.1 Preparation of Human CTLA-4 and Macaca fascicularis (Cynomolgus Monkey) CTLA-4 ECD Proteins

Human and Cynomolgus monkey (cyno) CTLA-4 extracellular domain (ECD) genes with hexa-histidine (6×His) or Fc-tag were cloned into expression vector pcDNA3.3, and then used for transfection of Expi293 cells (Invitrogen-A14527) by using Expi293 Expression System Kit (Invitrogen-A14524). The cells were cultured in Expi293 Expression Medium (Invitrogen-A1435101) on an orbital shaker platform rotating at 135 rpm, in a 37° C. incubator containing a humidified atmosphere with 8% CO₂. The harvested supernatant was used for protein purification. His-tagged proteins were purified using Ni-NTA column (GE healthcare-17-5247-01) and Fc-tagged proteins were purified using Protein A column (GE healthcare-17-5438-02).

1.1.2 Benchmark Antibodies

Reference Benchmark antibodies W316-BMK1

DNA sequences encoding the variable regions (VH and VL) of anti-CTLA-4 antibody Ipilimumab (sequences were based on clone 10D1 in U.S. Pat. No. 6,984,720 B1) were synthesized in Sangon Biotech (Shanghai, China), and then cloned into modified pcDNA3.4 expression vectors with constant region of human IgG1, or human IgG4. The plasmid containing VH and VL gene were co-transfected into Expi293 cells. Cells were cultured for 5 days and the supernatant was collected for antibody protein purification. The anti-CTLA-4 benchmark W316-BMK1 antibody of IgG1 format is designated in the following examples as “W316-BMK1” for short, unless otherwise indicated (e.g. as “W316-BMK1.IgG1”, in a few occasions to distinguish from its IgG4 counterpart “W316-BMK1.IgG4”).

1.1.3 Antibody Purification

Harvested cell culture supernatants containing antibody proteins were loaded to Protein A column after adjusting pH to 7.0. Antibodies were eluted by Glycine-HCl (pH 2.5) followed with immediately neutralization using 1 M Tris. Antibody concentration was measured by Nano Drop. The purity of proteins was evaluated by SDS-PAGE and HPLC-SEC.

1.2. Cell and Cell Line Preparation

1.2.1. Preparation of Engineered Cell Lines

CHO-K1 or 293F cells were transfected with the pcDNA 3.3. expression vectors containing gene encoding full length human CTLA-4, cyno CTLA-4, human CD80 or human CD86, respectively, by Lipofectamine 2000 (Invitrogen) or plasfect (Bioline). Cells were cultured in medium containing proper selection pressure. Human CTLA-4, CD80, CD86 high expression stable cell lines and Cyno CTLA-4 cell pool were selected by limited dilution.

1.2.2 Cultivation of Cell Lines

T-75 flasks and complete growth medium F12-K with 10% FBS and 8 μg/ml Blasticidin were used for subculture of CHO-K1 cell lines. Freestyle 293 Expression Medium with 8 μg/ml Blasticidin for 293F cell lines. Medium was renewed every 2 to 3 days and Trypsin-EDTA solution was used for detaching the CHO-K1 cells. For long term storage, the cells were frozen in complete growth medium supplemented with 5% (v/v) DMSO and stored in liquid nitrogen vapor phase.

1.3 Generation of VHH

1.3.1 Immunization

To induce a humoral immune response directed towards CTLA-4 in a Llama glama, the animal received alternate s.c. injection of CTLA-4 ECD protein for totally seven doses at one to two weeks interval. The dose ranged from 500 ug to 1000 ug per injection.

1.3.2 Serum Titer Detection

The CTLA-4 specific antibody titers in pre-immune and immune sera were monitored by ELISA. ELISA plates (Nunc, Rochester, Minn., USA) were coated with 1 μg/ml of CTLA-4 ECD protein and incubated overnight at 4° C. After blocking and washing, serial dilutions of pre-immune and immune sera were added and incubated at room temperature for 1 h, then followed with goat anti-Llama IgG-HRP (Novas Biologicals, Littleton, Colo., USA) at room temperature for 30 min. After washing, TMB substrate was added and the reaction was stopped by 2M HCl. The absorbance at 450 nm was read using a microplate reader (Molecular Device).

1.3.3 Phage Library Construction

7 days after the final injection, 50 ml blood was collected, and peripheral blood mononuclear cells (PBMC) were prepared by density gradient centrifugation on Ficoll-Hypaque (GE Healthcare, Little Chalfont, UK). 1×10⁷/ml PBMCs were incubated with biotinylated CTLA4 ECD protein coupled beads at 4° C. for 30 min and the cells binding to the beads were purified by MACS separation (Miltenyi Biotec). Total RNA from the selected cells was extracted with RNeasy Plus Mini Kit (QIAGEN) and transcribed into cDNA using SuperScript III First-Strand Synthesis SuperMix (Invitrogen). The purified cDNA was then used as template to amplify the repertoire of Ig heavy chain-encoding gene segments with the use of signal peptide domain specific primers and CH2 domain specific primers. This amplification resulted in PCR fragments of approximately 900 bp (representing conventional IgG) and 700 bp (representing heavy-chain only IgG that lack a CH1 domain). The two classes of heavy chain encoding genes were then size-separated on agarose gels and the genes encoding heavy-chain only IgG were purified by QIAquick Gel Extraction Kit (Qiagen, Hilden, Germany). The purified fragments were used as templates to amplify the VHH repertoire with the use of framework1 (FR1) and framework4 (FR4) specific primer pairs. This amplification procedure introduced a Sfi I restriction site at the 5′ end of FR1 and a Not I restriction site at the 3′ end of FR4. The repertoire of PCR-amplified VHH genes of about 300-400 bp were loaded on agarose gels and purified by QIAquick Gel Extraction Kit. The purified fragments were then cut with Sfi I and Not I and purified by QIAquick PCR Purification Kit (Qiagen, Hilden, Germany). The VHH gene fragments were finally ligated in phagemid vector pFL249 and electrotransformed into E. coli TG1. After transformation, the TG1 cells were cultured in SOC medium with shaking at 200 rpm for 1 h, then the E. coli TG1 were plated onto 2YT agar plates supplemented with 100 μg/mL Carb and 1% (w/v) glucose, and cultured at 37° C. overnight. The next day, the colonies were scraped into liquid 2YT medium supplemented with 1/3 (v/v) of 80% glycerol and were stored at −80° C.

1.3.4 Panning and Screening

To select VHH specific binding to human CTLA4, phage display panning on immobilized CTLA-4 ECD proteins was applied. Briefly, bug of CTLA-4 ECD proteins were coated onto an immunoplates (Nunc, Rochester, Minn., USA) in 1 ml coating buffer (Na₂CO₃/NaHCO₃, PH=9.2) overnight at 4° C. After blocking with 10% skim milk for 1 h, 1×10¹² library phage population was added and incubated at room temperature for 2 h. After 10 times washing with PBS containing 0.5% (v/v) Tween 20 (PBST), the nonspecifically adsorbed phage were discarded and the target specific phage were eluted by Glycine-HCl pH2.2 and then neutralized by 1M Tris-HCl pH8.0 for infection of exponential growth phaseTG1 cells, which then were incubated at 37° C. for 45 min without shaking. The infected TG1 cells were plated on 2YT agar plates and cultured overnight at 37° C. On the next day, the colonies were scraped off the plate with 3 ml 2YT and frozen at −80° C. by adding in 1/3 (v/v) 80% glycerol. The scraped bacteria libraries were inoculated into 2YT-Carb contain 100 μg/ml ampicillin and infected with helper phage M13KO7 in 2YT medium with 50 μg/ml kanamycin and 1 mM IPTG for phage rescue and used as input for the next round of panning.

After desired panning steps, phage infected TG1 cell colonies grown on the plates were scraped and pFL249 phagemid containing VHH fragments were extracted. The VHH expression vector were constructed by digestion of pFL249 plasmids with Sfi I and Not I and then ligated into linear expression vector pET-bac, which containing genes of hexa-histidine- and c-Myc-tag. The ligation products were transformed into E. coli BL21 (DE3) competent cells and then cultured in ZYM-5052 medium at 25° C. for 48 h with shaking at 180 rpm. The expression of his- and c-Myc-tag fused VHH protein in BL21 supernatants were screened for target specific binding by ELISA and FACS.

ELISA assay was used as the first screen method to test the binding of VHH E. coli culture supernatants to CTLA-4 ECD protein. Briefly, 96-well plates (Nunc) were coated with CTLA-4 ECD protein overnight at 4° C. After blocking and washing, the 3 times dilutions of E. coli supernatants were transferred to the coated plates and incubated at room temperature for 1 h. The plates were then washed and subsequently incubated with secondary antibody Goat Anti-c-Myc-HRP (Bethyl) for 1 h. After washing, TMB substrate was added and the color reaction was stopped by 2M HCl. The absorbance at 450 nm was read using a microplate reader (Molecular Device).

In order to confirm the native binding of CTLA-4 antibodies on conformational CTLA-4 molecules expressed on cell membrane, flow cytometry analysis was performed with CTLA-4 transfected 293F cell line and negative control parental 293F cell line. The cells were firstly incubated with the VHH E. coli supernatant samples in 96-well U-bottom plates (BD) at a density of 1×10⁵ cells/well at 4° C. for 1 h, then with the secondary antibody Mouse Anti-c-Myc-biotin (Sigma) at 4° C. for 30 min, followed by streptavidin PE (eBioscience) in the dark at 4° C. for 20 min. 2 times of washings were applied between each steps and the cells were resuspended in 1×PBS/1% BSA for flow cytometry (Intellicyt) analysis.

1.3.5 Sequencing

The positive E. coli clones selected by ELISA and FACS screening were sent to Biosune (Shanghai, China) for nucleotide sequencing of VHH gene. The sequencing results were analyzed using CLC Main Workbench (Qiagen, Hilden, Germany).

1.3.6 VHH Protein Production

The BL21 E. coli clones harboring VHH gene were cultured in 40 ml of ZYM-5052 medium at 25° C. for 48 h with shaking at 230 rpm. The expression of his- and c-Myc-tag fused VHH protein in BL21 supernatant was confirmed by SDS-PAGE, and then purified using Ni-NTA column. The purity of VHH was determined by SDS-PAGE and analytic SEC-HPLC. For low supernatant expression clones, ultrasonic (Scientz, Ningbo, China) breaking E. coli cells was used to release soluble VHH proteins and whole cell lysates were purified.

1.3.7 Chimeric VHH-Fc (hIgG1) Protein Production

The clones of interest were converted to VHH-Fc hIgG1 fusion antibodies. Briefly, the VHH genes were amplified from the pET-bac vectors using VHH-specific cloning primers containing appropriate restriction sites then cloned by fusion into a modified human Fc (IgG1) expression pcDNA3.3 vector to create corresponding clones of VHH-Fc hIgG1 chimeric antibody. Expi-293 cells were transiently transfected with the vector for chimeric antibody expression. The culture supernatant containing antibodies was harvested and purified using Protein A chromatography.

1.4 Antibody Humanization and PTM Removal

VHHs with high affinity and specificity to CTLA-4 were selected for humanization.

“Best Fit” approach was used to humanize VHH chains. Amino acid sequences of VHH framework regions were blasted against human germline V-gene database, and humanized VHH sequences were generated by replacing human CDR sequences in the top hit with VHH CDR sequences using Kabat CDR definition. Certain residues in the framework region were back-mutated to that of VHH in order to maintain the affinity. Humanized genes were back-translated, codon optimized for mammalian expression, and synthesized by GENEWIZ. These genes were re-amplified with cloning primers containing appropriate restriction sites and cloned into a modified pcDNA3.3 vector to express humanized VHHs linked with human IgG1 Fc region. Meanwhile, a post-translational modification (PTM) N-linked glycosylation residue N55 (kabat numbering) in CDR2 of W3166 was replaced with Q (N55Q, kabat numbering). After testing on CTLA-4 binding by SPR, two humanized and PTM removed clones W3166-z13 and W3166-z17 were obtained. W3166 VHH antibodies are fused with Fc of human IgG1 isotype, which are referred to herein as “W3166-z13” and “W3166-z17” for short.

1.5 In Vitro Characterization

1.5.1 Human CTLA-4-Binding (ELISA and FACS)

For ELISA binding, 96-well plates (Nunc) were pre-coated with 1.0 μg/ml in house made human CTLA4 ECD protein overnight at 4° C. After blocking with 2% BSA-PBS, serial diluted antibodies were added into each well and incubated for 1 hour at room temperature. HRP-labeled goat anti-human IgG (Bethyl A80-304P) were used as the secondary antibody and incubated for 1 hour. The color was developed by TMB substrate, and then stopped by 2N HCl. The absorbance was read at 450 nm using a Microplate Spectrophotometer (SpectraMax® M5e).

For FACS binding, engineered human CTLA-4 expressing cells were seeded at 1×10⁵ cells/well in U-bottom 96-well plates (COSTAR 3799). After centrifugation at 1500 rpm for 4 minutes at 4° C., the supernatants were moved and the serial dilutions of antibodies in 1% BSA-DPBS were added to the cells. The plates were incubated at 4° C. for 1 hour. After washing, PE-labeled goat anti-human IgG antibody (Jackson 109-115-098) was added and incubated at 4° C. for 1 hour. The binding of the antibodies to the cells was tested by flow cytometry and the mean fluorescence intensity (MFI) was analyzed by FlowJo.

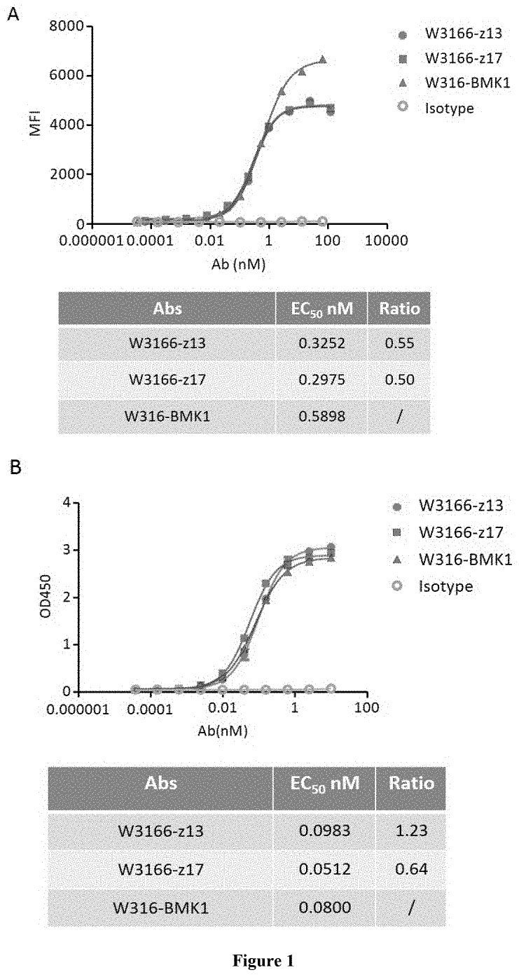

FIGS. 1A and 1B show that W3166-z13 and W3166-z17 bind to cell surface human CTLA-4 and immobilized human CTLA-4 ECD protein in a dose-dependent manner, respectively. W3166-z13 and W3166-z17 bind to cell surface human CTLA-4 with EC₅₀ values of 0.3252 nM and 0.2975 nM, respectively; in comparison, W316-BMK1 binds to cell surface human CTLA-4 with an EC₅₀ of 0.5898 nM. W3166-z13 and W3166-z17 bind to immobilized human CTLA-4 ECD protein with EC₅₀ values of 0.0983 nM and 0.0512 nM, respectively; in comparison, W316-BMK1 binds to immobilized human CTLA-4 ECD protein with an EC₅₀ of 0.0800 nM. The binding EC₅₀ of W3166-z13 and W3166-z17 are similar with that of W316-BMK1.

1.5.2 Cynomolgus CTLA-4-Binding (ELISA and FACS)

The binding of testing antibodies to cyno ELISA was assessed by ELISA and FACS as the described above. For ELISA binding, 96-well plates were coated with 1.0 μg/ml in house made cyno CTLA4 ECD protein. For FACS binding, engineered Cynomolgus CTLA4 expressing cell pool was used.

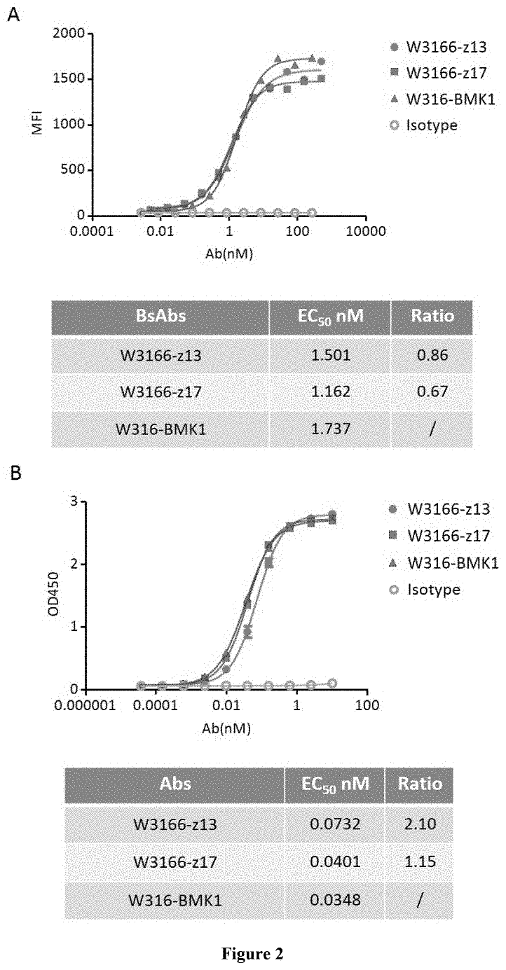

FIGS. 2A and 2B show that W3166-z13 and W3166-z17 bind to cell surface cyno CTLA-4 and immobilized cyno CTLA-4 ECD protein in a dose-dependent manner, respectively. W3166-z13 and W3166-z17 bind to cell surface cyno CTLA-4 with EC₅₀ values of 1.501 nM and 1.162 nM, respectively; in comparison, W316-BMK1 binds to cell surface cyno CTLA-4 with an EC₅₀ of 1.737 nM. W3166-z13 and W3166-z17 bind to immobilized cyno CTLA-4 ECD protein with EC₅₀ values of 0.0732 nM and 0.0401 nM, respectively; in comparison, W316-BMK1 binds to immobilized cyno CTLA-4 ECD protein with an EC₅₀ of 0.0348 nM. The binding EC₅₀ of W3166-z13 and W3166-z17 are comparable to that of W316-BMK1.

1.5.3 Competition ELISA

Competition ELISA was used to test whether W3166-z13 and W3166-z17 could block the binding of hCTLA4 to hCD80 or hCD86 protein.

Briefly, 96-well plates (Nunc) were coated with 1.0 μg/ml human CTLA4 ECD protein (in house) overnight at 4° C. After blocking with 2% BSA, serial dilutions of testing antibodies were pre-mixed with 0.25 μg/ml his-tagged human CD80 or CD86 protein (in house) and pipetted into each well and incubated for 1 hour at room temperature. After washing with PBST, biotin-labeled anti-His mAb (GenScript-A00613) was added and incubation 1 hour. After washing for 6 times, the bindings of hCD80 or hCD86 to hCTLA-4 were detected by Streptavidin-HRP (Lifetechnologies, #SNN1004). The color reaction was developed by TMB substrate, and then stopped by 2N HCl. The absorbance was read at 450 nm using a Microplate Spectrophotometer (SpectraMax® M5e).

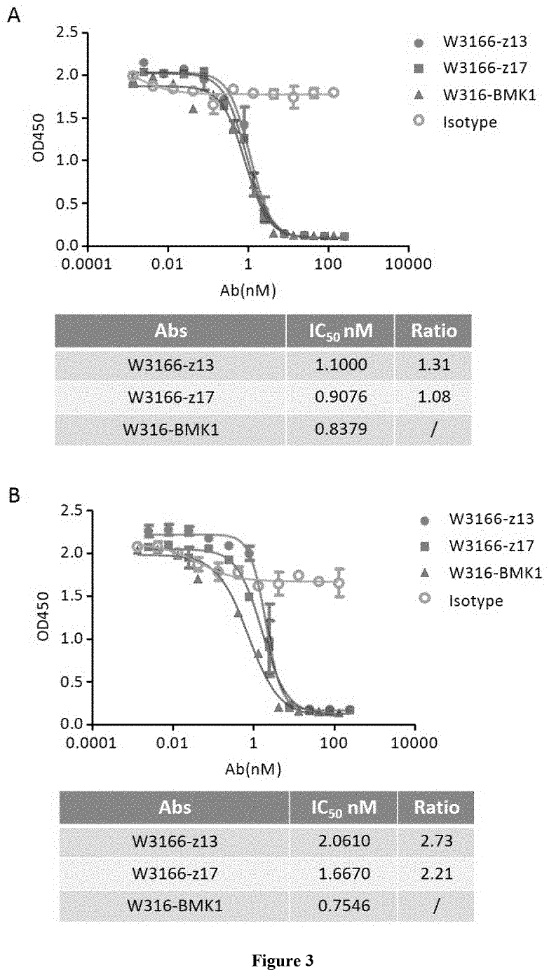

The results show that W3166-z13 and W3166-z17 block the binding of hCD80 to hCTLA-4 with IC₅₀ values of 1.1000 nM and 0.9076 nM, respectively, in comparison, W316-BMK1 blocks the binding of hCD80 to hCTLA-4 with an IC₅₀ of 0.8379 nM (FIG. 3A), and that W3166-z13 and W3166-z17 block the binding of hCD86 to hCTLA-4 with IC₅₀ values of 2.0610 nM and 1.6670 nM, respectively, in comparison W316-BMK1 blocks the binding of hCD86 to hCTLA-4 with an IC₅₀ of 0.7546 nM (FIG. 3B), examined by competition ELISA. As can be seen, W3166-z13 and W3166-z17 can block the binding of hCTLA4 to hCD80 or hCD86 protein as effectively as W316-BMK1.

1.5.4 Competition FACS

Competition FACS was used to test whether W3166-z13 and W3166-z17 could block hCTLA-4 binding to hCD80 or hCD86 on cell surface.

Human CD80 or CD86 transfected cells (in house) were added to 96-well plate (COSTAR 3799) at 1×10⁵ cell per well and centrifuged at 1500 rpm for 4 minutes at 4° C. before removing the supernatant. Serial dilutions of test antibodies in 1% BSA-DPBS were pre-mixed with biotinylated human CTLA4.ECD.protein (in house) and then the mixtures were added to the CD80 or CD86 expressing cells in the plate and incubated for 1 hour at 4° C. After washing, streptavidin PE (BD Pharmingen-554061) was added to the cells and incubated at 4° C. for 1 hour. The fluorescence values were measured by flow cytometry and analyzed by FlowJo.

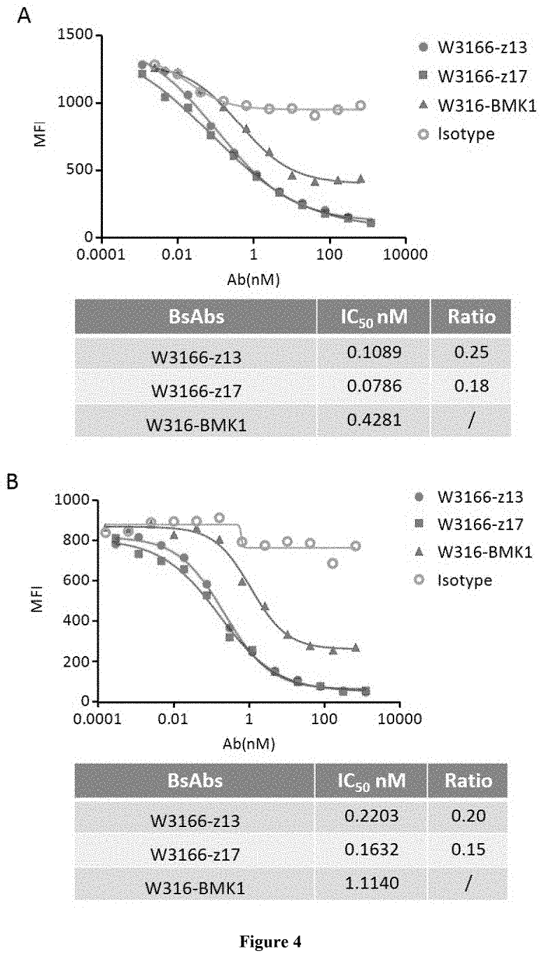

Results show that W3166-z13 and W3166-z17 block the binding of hCD80 to hCTLA-4 with IC₅₀ values of 0.1089 nM and 0.0786 nM, respectively, in comparison, W316-BMK1 blocks the binding of hCD80 to hCTLA-4 with an IC₅₀ of 0.4281 nM (FIG. 4A), and that W3166-z13 and W3166-z17 block the binding hCD86 of hCTLA-4 with IC₅₀ values of 0.2203 nM and 0.1632 nM, respectively, in comparison W316-BMK1 blocks the binding of hCD86 to hCTLA-4 with an IC₅₀ of 1.1140 nM (FIG. 4B), determined by competition FACS. FIGS. 4A and 4B show that W3166-z13 and W3166-z17 block the binding of hCTLA4 to cell surface hCD80 and hCD86 more effectively as compared with W316-BMK1.

1.5.5 Primary PBMC SEB Stimulation Assay

Human peripheral blood mononuclear cells (PBMCs) were freshly isolated from healthy donors using Ficoll-Paque (STEMCELL-07861) PLUS gradient centrifugation. Isolated PBMCs in complete RPMI-1640 (containing 10% FBS and 1% PS) were mixed with serial dilutions of W3166-z13 and W3166-z17 and SEB (Staphylococcal enterotoxin B) at the concentration of 0.1 ng/mL and added to 96-well round bottom plates in complete RPMI-1640 medium. The plates were incubated at 37° C., 5% CO₂. IL-2 and IFN-γ quantitation were determined on day 3 after incubation

Human IFN-γ and IL-2 were measured by ELISA using matched antibody pairs. Recombinant human IFN-γ (peprotech, 300-02-250UG) and recombinant human IL-2 (R&D-202IL) were used as standards, respectively. The plates were pre-coated with the capture antibody specific for human IFN-γ (Invitrogen, M700A) or IL-2 (R&D, MAB602). After blocking, standards or samples were added into the plates and incubated for 2 hours at room temperature. Following removal of the unbound substances, the biotin-conjugated detecting antibody specific for IFN-γ (Invitrogen, M701B) or IL-2 (R&D, BAF202) was added to the wells and incubated for 1 hour, followed by HRP-conjugated streptavidin for 30 minutes at room temperature. Washes were performed between each step. The color was developed by dispensing 100 μL of TMB substrate, and then stopped by 100 μL of 2N HCl. The absorbance was read at 450 nm using a microplate spectrophotometer (SpectraMax® M5e)

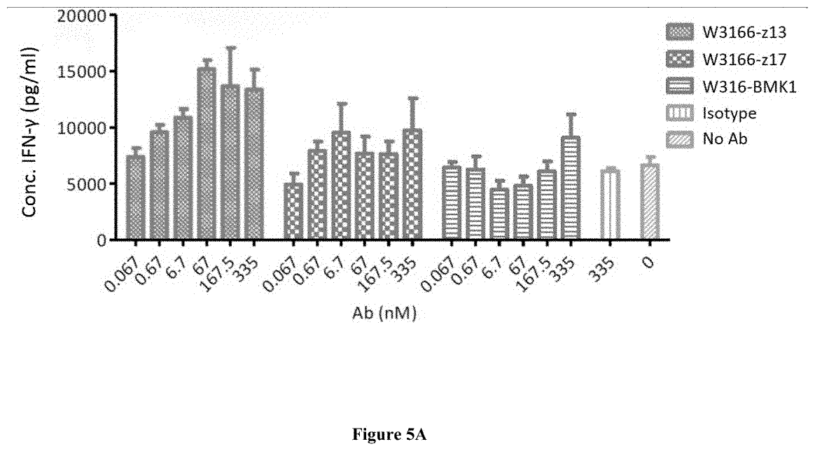

The results show that W3166-z13 and W3166-z17 enhance IFN-γ (FIG. 5A) and IL-2 (FIG. 5B) production in SEB stimulation assay with comparable potency to W316-BMK1.

1.5.6 Epitope Binning ELISA

The binding epitope of W3166-z13 and W3166-z17 was binned against W316-BMK1 by ELISA.

Briefly, 96-well plates (Nunc) were coated with 1.0 μg/ml human CTLA4 protein (in house) overnight at 4° C. After blocking with 2% BSA-PBS, pre-mixed serially diluted antibodies and 0.02 μg/ml W316-BMK1-Biotin were added and incubated for 1 hour at room temperature. After washing, HRP-conjugated streptavidin were added and incubated for 1 hour. The color was developed by dispensing TMB substrate, and then stopped by 2N HCl. The absorbance was read at 450 nm using a Microplate Spectrophotometer (SpectraMax® M5e).

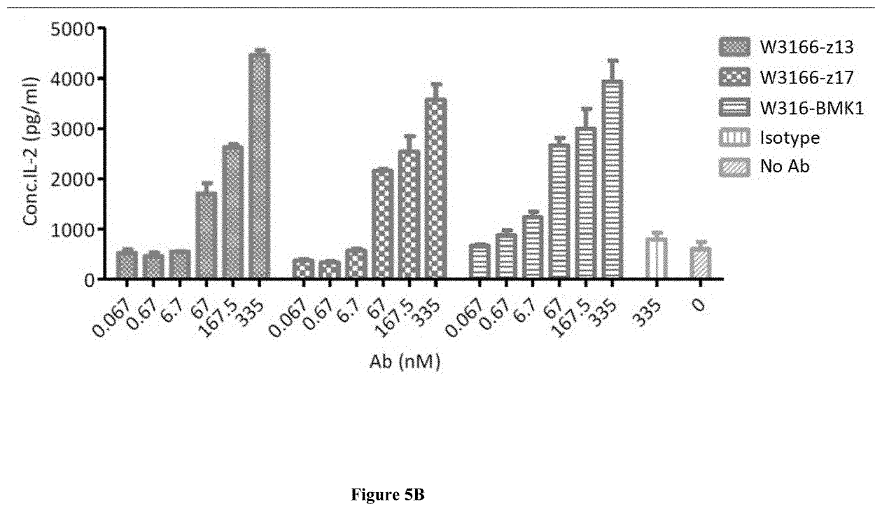

FIG. 6 shows that W3166-z13 and W3166-z17 have similar epitope binning with W316-BMK1.

1.5.7 Antibody-Dependent Cell-Mediated Cytotoxicity (ADCC) Assay

The ADCC assay was tested by using DELFIA® EuTDA Cytotoxicity Reagents (PerkinElmer AD0116). Briefly, 1×10⁵ per well of human CTLA4 transfected cells loaded by BATDA reagent were plated into 96-well plates containing serial dilutions of W3166-z13 and W3166-z17. Then PBMCs as effector cells were added into the plate at the effector/target ratio of 50:1. The plates were kept at 37° C. in a 5% CO₂ incubator for 4 hours. Target cell lysis was determined by DELFIA Europium Solution (Perkinelmer). The europium and the ligand form a highly fluorescent and stable chelate (EuTDA), which was read by SpectraMax® M5e.

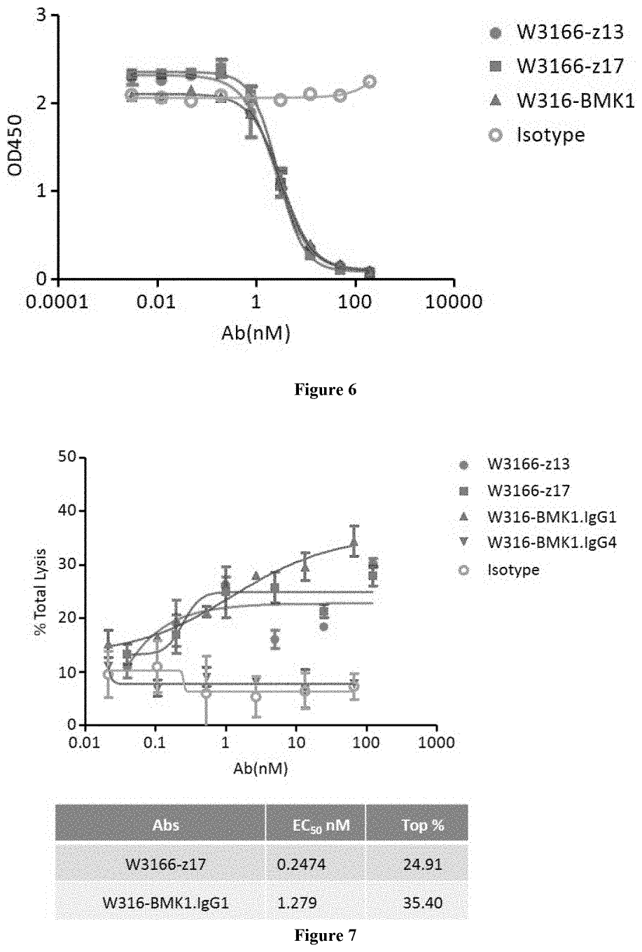

FIG. 7 shows that W3166-z13 and W3166-z17 induce ADCC effect on hCTLA4 transfected cells. Both IgG1 and IgG4 isotypes of benchmark antibody were used as references, designated as W316-BMK1.IgG1 and W316-BMK1.IgG4, respectively. As can be seen in FIG. 7, both W3166-z13 and W3166-z17 induce ADCC effect on hCTLA4 transfected cells. W3166-z17 showed an EC₅₀ of 0.2474 nM in inducing ADCC effect, while W316-BMK1.IgG1 showed an EC₅₀ of 1.279 nM in inducing ADCC effect.

1.5.8 Complement Dependent Cytotoxicity (CDC) Test

In house made engineered human CTLA4 expressing cells and serial dilutions of W3166-z13 and W3166-z17 were mixed and added into 96-well plates. Human complement was added at the dilution ratio of 1:50. The plate was kept at 37° C. in a 5% CO₂ incubator for 4 hours. Target cell lysis was determined by CellTiter-Glo (Promega). Rittman induced Raji cell lysis was used as positive control.

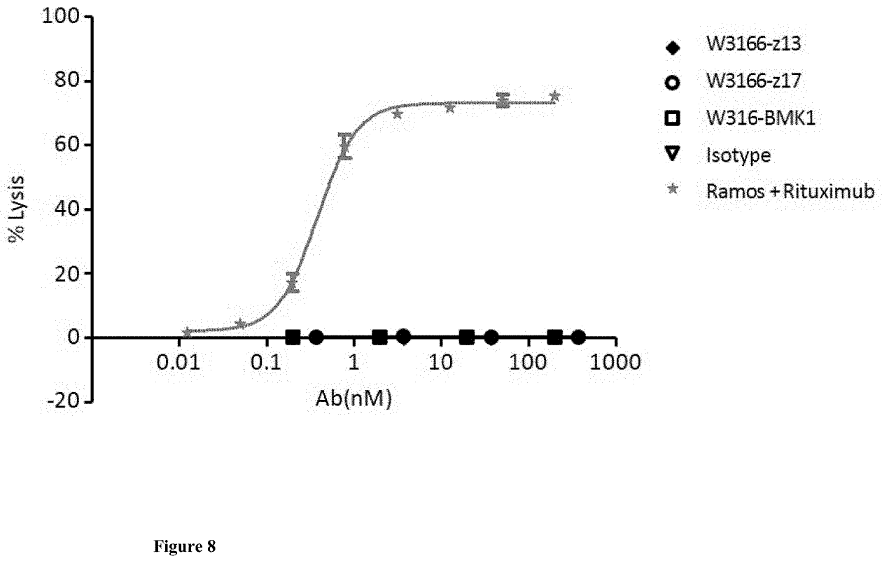

FIG. 8 shows that W3166-z13 and W3166-z17 do not induce CDC effect on hCTLA4 transfected cells.

1.5.9 Affinity Measured by FACS

Human CTLA-4 or cyno CTLA-4 transfected cells were plated into 96-well U-bottom plates (BD) at a density of 5×10⁴ cells/well. Testing antibodies were 1:2-fold serially diluted in 1% BSA-PBS and incubated with the cells at 4° C. for 1 hr. After centrifugation at 1500 rpm for 4 min, the supernatant was discarded. The secondary antibody FITC conjugated goat anti-human IgG Fc (Jackson Immunoresearch Lab) was added and incubated at 4° C. in the dark for 30 min. The cells were then washed once and re-suspended in 1% BSA-PBS for the analysis by flow cytometry (BD CantoII). Fluorescence intensity was converted to bound molecules/cell based on the quantitative beads (Quantum™ MESF Kits, Bangs Laboratories). K_{D} was calculated using Graphpad Prism5. Table 4 shows that W3166-z17 has better affinity K_{D} (M) to cell surface human CTLA4 than that of W316-BMK1.

TABLE 4
Affinity measured by FACS

| | W3166-z17 | WBP316-BMK1 |
|---|---|---|
| hPro1 | | |
| Bmax | 1.3E−10 | 1.5E−10 |
| K_{D} | 4.9E−11 | 2.8E−10 |
| r² | 0.99 | 0.99 |
| cPro1 | | |
| Bmax | 1.2E−10 | 1.1E−10 |
| K_{D} | 1.4E−10 | 2.8E−10 |
| r² | 0.99 | 0.98 |

1.5.10 Kinetic Binding Affinity Measured by Surface Plasmon Resonance (Via Biacore)

The experiment was to measure the on-rate constant (ka) and off-rate constant (kd) of the antibodies to CTLA-4 ECD based on SPR technology. The affinity constant (K_{D}) is thus consequently determined. Biacore T200, Series S Sensor Chip CM5, Amine Coupling Kit, and 10XHBS-EP were from GE Healthcare. Goat anti-human IgG Fc antibody was from Jackson ImmunoResearch Lab (catalog number 109-005-098). In immobilization step, the activation buffer was prepared by mixing 400 mM EDC and 100 mM NHS immediately prior to injection. The CM5 sensor chip was activated for 420 s with the activation buffer. 30 μg/mL of goat anti-human IgG Fc antibody in 10 mM NaAc (pH 4.5) was then injected to Fc1-Fc4 channels for 200 s at a flow rate of 5 μL/min. The chip was deactivated by 1 M ethanolamine-HCl (GE). Then the antibodies were captured on the chip. Briefly, 4 μg/mL antibodies in running buffer (HBS-EP+) was injected individually to Fc3 channel for 30 s at a flow rate of 10 μL/min. Eight different concentrations (20, 10, 5, 2.5, 1.25, 0.625, 0.3125 and 0.15625 nM) of analyte human CTLA-4 (W316.hprol.ECD.his) and blank running buffer were injected orderly to Fc1-Fc4 channels at a flow rate of 30 μL/min for an association phase of 120 s, followed by 2400 s dissociation phase. Regeneration buffer (10 mM Glycine pH 1.5) was injected at 10 μL/min for 30 s following every dissociation phase. Table 5 shows that the affinity of W3166-z17 to human CTLA-4 protein is similar with that of W316-BMK1.

TABLE 5
Kinetic binding affinity measured by surface
plasmon resonance (via Biacore)

| Analyte | Ligand | ka (1/Ms) | kd (1/s) | K_{D} (M) |
|---|---|---|---|---|
| Human CTLA-4 | W3166-z17 | 4.81E+05 | 1.50E−03 | 3.13E−09 |
| | W316-BMK1 | 3.61E+05 | 1.20E−03 | 3.32E−09 |

1.5.11 Human Serum Stability Test

Testing antibodies were incubated in freshly isolated human serum (serum content >90%) at 37° C. On indicated time points, an aliquot of serum treated sample was removed from the incubator and snap frozen in liquid nitrogen, and then stored at −80° C. until test. The samples were quickly thawed immediately prior to the stability test. Serial dilutions of antibodies were incubated with CTLA-4 transfected cells for 1 hour at 4° C. After washing, FITC conjugated goat anti-human IgG antibody (Jackson ImmunoResearch) was added to the cells and incubated at 4° C. for 1 hour. Finally, the cells were washed and resuspended in 1% BSA-PBS. The MFI fluorescence values were measured by flow cytometry and analyzed by FlowJo.

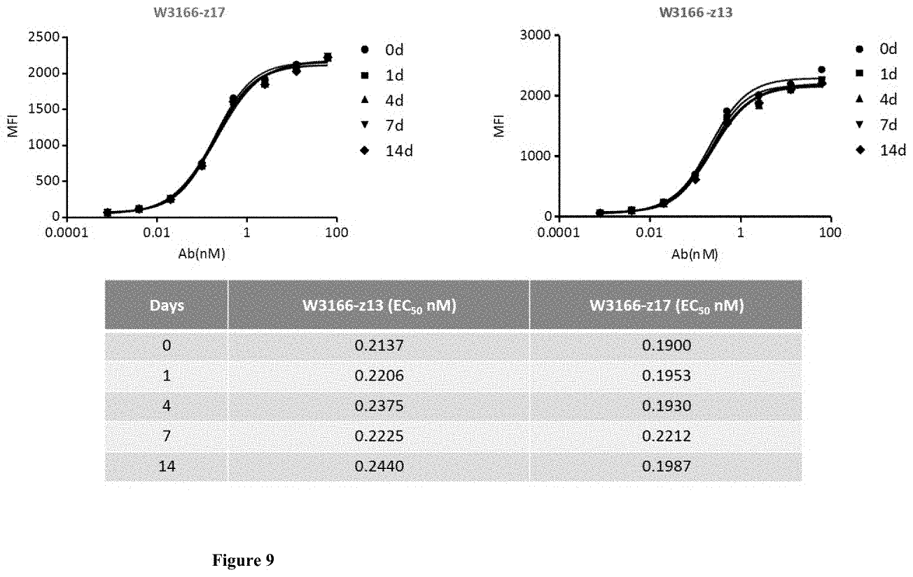

As can be seen in FIG. 9, W3166-z13 and W3166-z17 show consistent EC₅₀ values ranging from 0.2137-0.2440 nM and 0.1900-0.2212 nM, respectively, throughout the tested period (0-14 days), demonstrating that they are stable in human serum stability test.

1.5.12 Cross-Family Binding Test

96-well plates (Nunc) were pre-coated with 1.0 μg/ml of hCTLA-4.His, hICOS.mFC, hCD28.mFc, hBTLA.His and hPD-1.mFc overnight at 4° C., respectively. After blocking with 2% BSA-PBS, 10 μg/ml and 1.0 μg/ml of testing antibodies were respectively added into the plates and incubated for 1 hour at room temperature. The secondary antibody HRP-labeled goat anti-human IgG (Bethyl A80-304P) was then added and incubated for 1 hour. Washes with PBST were performed between each step. The color reaction was developed by TMB substrate, and then stopped by 2N HCl. The absorbance was read at 450 nm using a Microplate Spectrophotometer (SpectraMax® M5e).

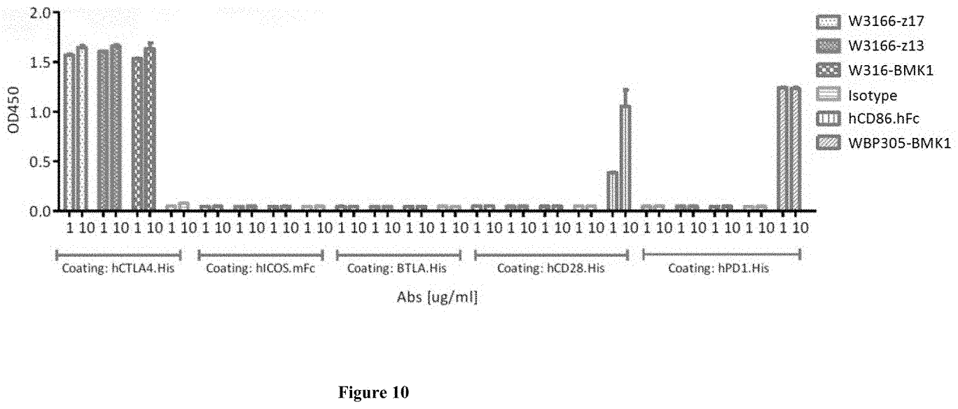

FIG. 10 shows that W3166-z13 and W3166-z17 specifically bind to human CTLA-4 and do not cross-react with hICOS, BTLA, hCD28 and hPD1.

1.5.13 Non-Specific Binding Test (FACS)

Various human tumor cell lines were used for non-specific binding test by FACS. Briefly, the viable cells were centrifuged at 1500 rpm for 4 min and then re-suspended in an appropriate volume of 1% BSA-PBS to the concentration of 1×10⁶ cell/ml. 100 μl cell suspension was added into each well of 96-well U-plate. After centrifugation, the cells were re-suspended with 100 μl/well diluted W3166-z17 and control isotype antibodies at 10 μg/ml in 1% BSA-PBS. After incubation at 4° C. for 1 hour, the cells were washed twice with 1% BSA-PBS and then incubated with 5 μg/ml goat anti-human IgG Fc-PE (Jackson, 109-115-098 & 126973) at 4° C. for 30 min. After two times of washing, the cells were re-suspended in 100 μl/well 1% BSA-PBS and kept at 4° C. in the dark until FACS analysis (BD Canto II).

The results show that W3166-z17 has no non-specific binding (Table 6). An antibody of IgG1 Kappa isotype and an antibody of IgG1 Lamda isotype that do not bind to CTLA-4 were used as isotype controls. A PE-labeled goat anti-human antibody was used as the 2^{nd} antibody only control.

TABLE 6

| | MFI | | | | | | | | | | | |
|---|---|---|---|---|---|---|---|---|---|---|---|---|
| | | | | | | | | | | | | CHO- |
| | Ramos | Jurkat.2B8 | Hut78 | A431 | A204 | CaLu-6 | A375 | HepG2 | BxPC-3 | HT29 | FaDu | K1 |
| W3166-z17 | 69 | 53 | 33 | 30 | 40 | 45 | 56 | 43 | 40 | 36 | 48 | 42 |
| IgG1 Kappa | 45 | 37 | 75 | 36 | 33 | 49 | 32 | 32 | 41 | 27 | 33 | 29 |
| isotype control | | | | | | | | | | | | |
| IgG1 Lamda | 44 | 26 | 30 | 27 | 32 | 36 | 38 | 33 | 36 | 27 | 33 | 34 |
| isotype control | | | | | | | | | | | | |
| Blank | 28 | 23 | 27 | 25 | 31 | 32 | 30 | 30 | 32 | 25 | 23 | 28 |
| NC1 (Goat-Anti- | 42 | 21 | 26 | 25 | 31 | 32 | 28 | 30 | 33 | 26 | 23 | 28 |
| Human PE) | | | | | | | | | | | | |

1.5.14 Thermal Stability by DSF Assay

A DSF assay was performed using Real-Time Fluorescent Quantitative PCR (QuantStudio 7 Flex, Thermo Fisher Scientific). Briefly, 19 μL of antibody solution was mixed with 1 μL of 62.5×SYPRO Orange solution (Invitrogen) and added to a 96 well plate (Biosystems). The plate was heated from 26° C. to 95° C. at a rate of 2° C./min, and the resulting fluorescence data were collected. The negative derivatives of the fluorescence changes with respect to different temperatures were calculated, and the maximal value was defined as melting temperature Th. If a protein has multiple unfolding transitions, the first two Tₘ were reported, named as Tₘ₁ and Tₘ₂. Tₘ₁ is always interpreted as the formal melting temperature Tₘ to facilitate comparisons between different proteins. Data collection and Tₘ calculation were conducted automatically by its operation software (QuantStudio Real-Time PCR PCR Software v1.3).

Table 7 shows the result of DSF test. W3166-z17 has Tm of 54.1°C.

TABLE 7

| Protein | | | | Concentration | |
|---|---|---|---|---|---|
| Name | Isotype | pl | Buffer | (mg/ml) | Tₘ(° C.) |
| W3166-z17 | higG1 | 8.60 | PBS | 4.7 | 54.1 |

### SEQUENCE LISTING

The sequence listing submitted herewith in the ASCII text file entitled “SEQUENCE LISTING127501005US1,” created Sep. 15, 2020, with a file size of 6,500 bytes, is incorporated herein by reference in its entirety.

1. (canceled)

2. An antibody polypeptide, comprising a heavy chain variable domain that specifically binds to CTLA-4, wherein the heavy chain variable domain comprises a) CDR1 of SEQ ID NO: 1, CDR2 of SEQ ID NO: 10 and CDR3 of SEQ ID NO: 3; or b) CDR1 of SEQ ID NO: 1, CDR2 of SEQ ID NO: 2 and CDR3 of SEQ ID NO: 3.

3. The antibody polypeptide of claim 2, wherein the heavy chain variable domain comprises a sequence selected from the group consisting of SEQ ID NOs: 4, 6 and 8, and a homologous sequence thereof having at least 80% sequence identity yet retaining specific binding affinity to CTLA-4.

4. The antibody polypeptide of claim 2, further comprising one or more amino acid residue substitutions or modifications yet retaining specific binding affinity to CTLA-4.

5. (canceled)

6. (canceled)

7. The antibody polypeptide of claim 2, wherein the heavy chain variable domain is derived from a VHH domain.

8. The antibody polypeptide of claim 2, further comprising an immunoglobulin constant region, optionally a constant region of human Ig, or optionally a constant region of human IgG1.

9. (canceled)

10. (canceled)

11. The antibody polypeptide of claim 2, capable of specifically binding to human CTLA-4 at an EC50 value of no more than 0.5 nM as measured by flow cytometry.

12. (canceled)

13. The antibody polypeptide of claim 2, capable of specifically binding to Cynomolgus monkey CTLA-4, and/or mouse CTLA-4.

14. (canceled)

15. (canceled)

16. (canceled)

17. A pharmaceutical composition comprising the antibody polypeptide of claim 2 and a pharmaceutically acceptable carrier.

18. A polynucleotide encoding the antibody polypeptide of claim 2.

19. The polynucleotide of claim 18, comprising a nucleotide sequence selecting from a group consisting of SEQ ID NO: 5, SEQ ID NO: 7 and SEQ ID NO: 9, and/or a homologous sequence thereof having at least 80% sequence identity, and/or a variant thereof having only degenerate substitutions.

20. A vector comprising the polynucleotide of claim 18.

21. A host cell comprising the vector of claim 20.

22. A method of expressing the antibody polypeptide of claim 2, comprising culturing the host cell of claim 21 under the condition at which the vector of claim 20 is expressed.

23. A method of treating a disease or condition in a subject that would benefit from modulation of CTLA-4 activity, comprising administering to the subject a therapeutically effective amount of the antibody polypeptide of claim 2 or the pharmaceutical composition of claim 17.

24. The method of claim 23, wherein the disease or condition is cancer, autoimmune disease, inflammatory disease, infectious disease, graft versus host disease (GVHD), or transplant rejection.

25. (canceled)

26. The method of claim 24, wherein the cancer is lymphoma, bladder cancer, bone cancer, brain and central nervous system cancer, breast cancer, uterine or endometrial cancer, rectal cancer, esophageal cancer, head and neck cancer, anal cancer, gastrointestinal cancer, intra-epithelial neoplasm, kidney or renal cancer, leukemia, liver cancer, lung cancer, melanoma, myeloma, pancreatic cancer, prostate cancer, sarcoma, skin cancer, squamous cell cancer, stomach cancer, testicular cancer, vulval cancer, cancer of the endocrine system, cancer of the parathyroid gland, cancer of the adrenal gland, penile carcinoma, solid tumors of childhood, tumor angiogenesis, spinal axis tumor, pituitary adenoma, or epidermoid cancer.

27. The method of claim 23, wherein the disease or condition is an environmentally induced cancer induced by asbestos or hematologic malignancies, wherein said cancer is selected from multiple myeloma, B-cell lymphoma, Hodgkin lymphoma, primary mediastinal B-cell lymphoma, non-Hodgkin's lymphoma, acute myeloid lymphoma, chronic myelogenous leukemia, chronic lymphoid leukemia (CLL), follicular lymphoma, diffuse large B-cell lymphoma (DLBCL), Burkitt's lymphoma, immunoblastic large cell lymphoma, precursor B-lymphoblastic lymphoma, mantle cell lymphoma, acute lymphoblastic leukemia (ALL), mycosis fungoides, anaplastic large cell lymphoma, T-cell lymphoma, and precursor T-lymphoblastic lymphoma, and any combinations of said cancers.

28. (canceled)

29. (canceled)

30. (canceled)

31. (canceled)

32. A method of diagnosing a CTLA-4 related disease or condition in a subject, comprising: a) contacting a sample obtained from the subject with the antibody polypeptide of claim 2; b) determining presence or amount of CTLA-4 in the sample; and c) correlating the presence or the amount of CTLA-4 to existence or status of the CTLA-4 related disease or condition in the subject.

33. (canceled)

34. (canceled)

35. A kit comprising the antibody polypeptide of claim 2, useful in detecting CTLA-4.


SEQUENCE LISTING (10 sequences)

SEQ ID NO: 1 - protein, 10 aa, Lama glama
GRTFSSYAMG

SEQ ID NO: 2 - protein, 17 aa, Lama glama
SIRWSDNTTYVPNSVKG

SEQ ID NO: 3 - protein, 17 aa, Lama glama
GPTRLSFYSGNYRTYDS

SEQ ID NO: 4 - protein, 126 aa, Lama glama
QVQLVESGGGLVQAGGSLRLSCAASGRTFSSYAMGWFRQAPGMEREFVASIRWSDNTTYVPNSVKGRFTISRDNAKNTVYLQMNTLKPEDTAVYYCATGPTRLSFYSGNYRTYDSWGQGTLVTVSS

SEQ ID NO: 5 - DNA, 378 bp, Lama glama
caggtgcagctcgtggagtctgggggaggattggtgcaggctgggggctctctgagactctcctgtgcggcctctggacgcaccttcagtagctatgccatgggctggttccgccaggctccagggatggagcgtgagtttgtagcatctattaggtggagtgataatacgacatacgtccctaactccgtgaagggccgattcaccatctccagagacaacgccaagaacacggtgtatctgcaaatgaacaccctgaaacctgaggacacggccgtttattactgtgcaacagggcccacgagactatcattttatagtggtaattatagaacttatgactcctggggccaggggaccctggtcaccgtctcctca

SEQ ID NO: 6 - protein, 126 aa, Artificial Sequence
  Synthetic
QVQLVESGGGLVQPGGSLRLSCAASGRTFSSYAMGWFRQAPGMEREFVASIRWSDQTTYVPNSVKGRFTISRDNSKNTLYLQMNSLRPEDTAVYYCATGPTRLSFYSGNYRTYDSWGQGTLVTVSS

SEQ ID NO: 7 - DNA, 378 bp, Artificial Sequence
  Synthetic
caggtgcagctggtggagagcggaggcggactggtgcagcctggaggaagcctgagactgagctgcgccgccagcggcagaaccttcagcagctacgccatgggctggttcagacaggcccctggcatggagagagagttcgtggccagcatcaggtggtccgaccagaccacctacgtgcccaacagcgtgaagggcaggttcaccatcagcagggacaacagcaagaacaccctgtacctccagatgaacagcctgagacccgaggataccgccgtgtactattgcgccaccggccccaccagactgagcttctacagcggcaactacaggacctacgacagctggggccagggaaccctggtgaccgtgagcagc

SEQ ID NO: 8 - protein, 126 aa, Artificial Sequence
  Synthetic
QVQLVESGGGVVQPGGSLRLSCAASGRTFSSYAMGWFRQAPGKEREFVASIRWSDQTTYVPNSVKGRFTISRDNSKNTLYLQMNSLRPEDTAVYYCATGPTRLSFYSGNYRTYDSWGQGTLVTVSS

SEQ ID NO: 9 - DNA, 378 bp, Artificial Sequence
  Synthetic
caggtgcagctggtggagagcggaggcggagtggtgcagcctggaggaagcctgagactgagctgcgccgccagcggcagaaccttcagcagctacgccatgggctggttcagacaggcccctggcaaggagagagagttcgtggccagcatcaggtggtccgaccagaccacctacgtgcccaacagcgtgaagggcaggttcaccatcagcagggacaacagcaagaacaccctgtacctccagatgaacagcctgagacccgaggataccgccgtgtactattgcgccaccggccccaccagactgagcttctacagcggcaactacaggacctacgacagctggggccagggaaccctggtgaccgtgagcagc

SEQ ID NO: 10 - protein, 17 aa, Artificial Sequence
  Synthetic
SIRWSDQTTYVPNSVKG